(19) United States
(12) Patent Application Publication
(10) Pub. No.: US 2008/0221551 A1
(43) Pub. Date: Sep. 11, 2008

(54) ACUTE KIDNEY INJURY TREATMENT SYSTEMS AND METHODS

(75) Inventors: Harry B. Goodson, Fremont, CA (US); Jeffrey M. Elkins, Woodside, CA (US); Samir R. Patel, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: FlowMedica, Inc., Fremont, CA (US)

(21) Appl. No.: 12/044,373

(22) Filed: Mar. 7, 2008

Related U.S. Application Data
(60) Provisional application No. 60/894,075, filed on Mar. 9, 2007.

(51) Int. Cl. A61M 31/00; A61M 25/00

(52) U.S. Cl. 604/508; 604/284

(57) ABSTRACT

Systems and methods for the clinical treatment of individuals susceptible to or suffering from acute kidney injury are provided. Drugs, biologics, or other therapies or treatments are administered by fluid agent delivery directly to the kidneys via their arterial blood supply. Bifurcated renal artery infusion catheter devices and methods can treat kidney injury in patients with locally-delivered drugs, biologics, and other agents, so as to increase the ability of the kidneys to excrete nitrogenous waste products from the blood, and prevent or ameliorate azotemia.

### CROSS-REFERENCES TO RELATED APPLICATIONS

This application is a nonprovisional of, and claims the benefit of the filing date of U.S. Provisional. Patent Application No. 60/894,075 filed Mar. 9, 2007, the entire disclosure of which is incorporated herein by reference for all purposes.

### FIELD OF THE INVENTION

Embodiments of the present invention relate generally to the clinical treatment of individuals susceptible to or suffering from acute kidney injury due to any number of causes. More particularly, embodiments of the present invention encompass fluid treatment or therapy directly to the renal arteries via a bifurcated renal artery infusion catheter system, where the treatment can be based on specific etiologies, severities, and degrees of progression of kidney injury.

### BACKGROUND OF THE INVENTION

Acute kidney injury (AKI) can be characterized by an abrupt decrease in the ability of the kidneys to excrete nitrogenous waste products from the blood, resulting in azotemia. Exemplary consensus definitions regarding the stages of AKI have recently been published by the Acute Dialysis Quality Initiative (ADQI) group (Bellolo, R. et al, Crit Care 8:R204-12 (2004)). The ADQI definitions involve what is known as the RIFLE criteria, which rank the progression of degree of kidney injury into five categories: Risk, Injury, Failure, Loss, and End-stage. Despite certain consensus agreements on revised definitions and suggestions for further research, consensus guidelines for effective AKI prevention and treatment have not been presented (Bellolo, R. et al, Crit Care 8:R204-12 (2004)). More recently, the Acute Kidney Injury Network (AKIN) has published revised guidelines (the AKIN criteria), based on the original RIFLE classification, but reflecting newer research suggesting that even smaller pertubations of renal function are indeed clinically relevant (Mehta, R. et al, Crit Care 11:R31-8 (2007)).

AKI may be pre-renal, intrinsic, or post-renal in origin, and may be hospital-acquired (generally iatrogenic) or patients may present for medical attention with the condition. A breakdown of AKI incidence by cause was recently published (Mehta, R. L. et al., Kidney Int 66:1613-21 (2004)). Etiologies or leading to AKI (those occurring in at least 5% of patients studied) include non-specific acute tubular necrosis (ATN), hypotension, sepsis, nephrotoxic ARF due to radiocontrast agents, congestive heart failure (CHF), hypovolemia, hepatorenal syndrome, cardiogenic shock, and hemorrhage.

Typically, length of stay (LOS), morbidity, mortality, and costs are all increased in patients developing AKI in-hospital. A recent review and analysis of over 9,000 patients by Chertow et al demonstrated increased risk of death (multivariate adjusted odds ratio (OR) of 4.4 for an increase of Cr of 50% over baseline) and increased costs ($5,510 US mean adjusted marginal increase for the same 50% increase in Cr). In this analysis, the development of an acute kidney injury was significantly associated with increased hospital length of stay (LOS) (Chertow, G. M. et al., J Am Soc Neptol 16:3365-70, (2005)). Similarly, in a study of over 2,000 post-coronary artery bypass grafting (CABG) patients by Mangano et al, those developing post-op AKI but not requiring dialysis demonstrated a high 19% in-hospital mortality rate, while those developing AKI requiring dialysis demonstrated a staggering 63% in-hospital mortality (Mangano, C. M. et al., Ann Intern Med 128(3):194-203 (1998 Feb. 1)).

Approximately 50% of hospital-acquired AKI is pre-renal in nature, being caused by renal hypoperfusion, and it is this type of AKI that may have the greatest chance of being readily and rapidly reversible. The etiology of pre-renal AKI may include any of those items identified by Mehta et al (see above), or others; however, in any event, the underlying mechanism often involves a substantial reduction in renal blood flow, leading to a reduction in kidney function due to tissue ischemia and if left untreated, eventual cellular death (necrosis).

Thus, there exists a need for systems and methods for preventing, treating, or ameliorating this deterioration in the renal blood supply, or alternatively for intervening quickly enough to restore proper blood flow prior to the onset of necrosis. Relatedly, there exists a need to improve or increase the ability of a patient's kidneys to excrete nitrogenous waste products from the blood, and thus prevent or ameliorate azotemia. Embodiments of the present invention address at least some of these needs.

### BRIEF SUMMARY OF THE INVENTION

Embodiments of the present invention relate to the clinical treatment of individuals susceptible to or suffering from acute kidney injury due to any number of causes. Embodiments encompass systems, devices, and means for providing drug, biologic, or other therapy or treatment comprising fluid agent delivery directly to the kidneys via their arterial blood supply. In some cases, embodiments of the invention provide a bifurcated renal artery infusion catheter device and method for its use in treating kidney injury in patients with locally-delivered drugs, biologics, and other agents. In some cases, embodiments of the present invention provide specific drug, biologic, and other agent treatment regimes for specific etiologies, severities, and degrees of progression of kidney injury. For example, a treatment method can include delivering a therapeutic agent from a therapeutic agent source through a bifurcated renal artery infusion catheter and into the first and second renal arteries, where the therapeutic agent is selected based on the stage or severity of the acute kidney injury, the cause of the acute kidney injury, or both.

Embodiments of the present invention encompass treatment for patients in at least the first three categories of AKI—Risk, Injury, and Failure by the RIFLE criteria, or Stage I, II, and III, by the AKIN criteria. Advantageously, such treatments can be provided to patients developing AKI in-hospital, where in most cases the insult that led to the AKI is known (e.g., surgical procedure, toxic insult), and thus the time course of the condition is known. Treatment can be provided to patients where permanent damage has not yet occurred to the point that kidney function cannot be at least partially recovered. Often, such patients respond where treatment is enacted in a timely manner. TRT can be used to treat or prevent acute kidney injury, particularly where patients are at risk of developing or are suffering from kidney injury related to sepsis, post-operative, renal cancers, and the like. TRT can also be used to improve symptoms and reduce complications associated with dialysis. Embodiments also encompass methods of monitoring patients prior to and in response to protocols for treating or preventing acute kidney injury. Similarly, embodiments encompass methods for decreasing symptoms or complications associated with acute kidney injury, and for improving indicators associated with kidney health. Embodiments also include methods for determining when to administer preventative or ameliorative treatments for acute kidney injury, as well as methods for evaluating and establishing regimens or protocols for preventing or inhibiting acute kidney injury.

In one aspect, embodiments of the present invention encompass methods for treating a patient suffering from acute kidney injury. An exemplary method may include placing a bifurcated renal artery infusion catheter within the abdominal aorta of the patient. The bifurcated infusion catheter can include a first renal delivery member with a first port and a second renal delivery member with a second port. The method may also include placing the first renal delivery member within a first renal artery of the patient, placing the second renal delivery member within a second renal artery of the patient, and delivering an amount of a therapeutic agent from a therapeutic agent source through the bifurcated renal artery infusion catheter and into the first and second renal arteries via the first and second ports, respectively. The acute kidney injury can be characterized by one or more clinical criteria or conditions. For example, the acute kidney injury can be characterized by an increase in serum creatinine by at least 50% over baseline, an absolute increase in serum creatinine of at least 0.3 mg/dL over baseline, a reduction in glomerular filtration rate of at least 25% compared to baseline, a decrease in urine output to 0.5 ml per kilogram of body weight or less per hour persisting for at least 6 hours, or any combination thereof. An acute kidney injury may be caused by or associated with exposure to a toxic agent such as a radiocontrast media, a non-steroidal anti-inflammatory drug (NSAID), or a chemotherapy agent. An acute kidney injury may also involve a pre renal kidney injury caused by or associated with a reduced cardiac output leading to reduced overall blood flow to the kidneys, trauma, reduced blood oxygenation, systemic toxicity caused by reaction to injury in another organ, systemic hypotension resulting from cardiorenal syndrome or acute decompensated heart failure, a reduction in circulating volume due to hemorrhage, a surgical procedure, or a reduction in local renal blood flow resulting from hepatorenal syndrome. When the acute kidney injury can be characterized according to such criteria, or when the acute kidney injury is caused or associated with by such conditions, the method may involve administration of a therapeutic agent that includes a vasodilator, an antioxidant, or both. The vasodilator can be fenoldopam mesylate or an analog or derivative thereof, dopamine or an analog or derivative thereof, or a prostaglandin or analog or derivative thereof, for example. The antioxidant can be ascorbic acid, sodium bicarbonate, or acetylcysteine, for example.

In a related aspect, an acute kidney injury can be characterized by an increase in serum creatinine by at least 100% over baseline, a reduction in glomerular filtration rate of at least 50% compared to baseline, a decrease in urine output to 0.5 ml per kilogram of body weight or less per hour persisting for at least 12 hours, or any combination thereof. When the acute kidney injury can be characterized according to such criteria, or when the acute kidney injury is caused by etiological conditions described herein, the method may involve administration of a therapeutic agent that includes a vasodilator, an antioxidant, an anti inflammatory agent, an antibiotic agent, or any combination thereof. The vasodilator can be fenoldopam mesylate or an analog or derivative thereof, dopamine or an analog or derivative thereof, or a prostaglandin or analog or derivative thereof, for example. The antioxidant can be ascorbic acid, sodium bicarbonate, or acetylcysteine, for example. The anti inflammatory agent can be a P 38 kinase inhibitor, for example. The antibiotic can be a bactericidal agent and a bacteriostatic agent, for example.

In a related aspect, an acute kidney injury can be characterized by an increase in serum creatinine by at least 200% over baseline, an absolute serum creatinine rise of at least 0.5 mg/dL to a value of at least 4 mg/dL, a reduction in glomerular filtration rate of at least 75% compared to baseline, a decrease in urine output to 0.3 ml per kilogram of body weight or less per hour persisting for at least 24 hours, a decrease in total urine output over 12 hours to 200 ml or less, or any combination thereof. When the acute kidney injury can be characterized according to such criteria, or when the acute kidney injury is caused by etiological conditions described herein, the method may involve administration of a therapeutic agent that includes a vasodilator, an antioxidant, a diuretic, an anti inflammatory agent, an antibiotic, a neurohormonally active agent, or any combination thereof. The vasodilator can be fenoldopam mesylate or an analog or derivative thereof, dopamine or an analog or derivative thereof, or a prostaglandin or analog or derivative thereof, for example. The antioxidant can be ascorbic acid, sodium bicarbonate, or acetylcysteine, for example. The diuretic can be hydrochlorothiazide (HCTZ), spironolactone, or a loop diuretic, for example. The antibiotic can be a bactericidal agent or a bacteriostatic agent, for example. The neurohormonally active agent can be a natriuretic peptide or an analog or derivative thereof, for example. In some cases, the loop diuretic may be furosemide. In some cases, the natriuretic peptide may be A type natriuretic peptide, B-type natriuretic peptide, C-type natriuretic peptide, a synthetic natriuretic peptide, or a bio-engineered natriuretic peptide.

In another aspect, an acute kidney injury can be caused at least in part by a kidney tumor or cancer. The tumor or cancer may include a renal cell carcinoma. When the acute kidney injury is caused by such conditions, the method may involve administration of a therapeutic agent that includes a vasodilator, an antioxidant, an anti inflammatory agent, a cytokine, a neurohormonally-active agent, or any combination thereof. The vasodilator can be fenoldopam mesylate or an analog or derivative thereof, dopamine or an analog or derivative thereof, or a prostaglandin or analog or derivative thereof, for example. The antioxidant can be ascorbic acid, sodium bicarbonate, or acetylcysteine, for example. The cytokine can be a lymphokine, for example. The neurohormonally active agent can be a natriuretic peptide or an analog or derivative thereof, for example. In some cases, the lymphokine can be interleukin-2 or a genetically engineered or modified version thereof, for example. In some cases, the natriuretic peptide can be A-type natriuretic peptide, B-type natriuretic peptide, C-type natriuretic peptide, a synthetic natriuretic peptide, or a bio engineered natriuretic peptide, for example.

In a further aspect, embodiments of the present invention encompass systems for treating a patient suffering from acute kidney injury. An exemplary system may include a bifurcated renal artery infusion catheter, a therapeutic agent source located externally to the patient, and a mechanism such as an infusion pump that delivers the therapeutic agent through the bifurcated renal artery catheter and directly into the right and left renal arteries of the patient. The bifurcated renal infusion catheter can be placed via a percutaneous means, and can include a distal bifurcated portion and a non-bifurcated proximal tubular portion. The infusion catheter can also include a catheter inner lumen traversing a length of the non-bifurcated proximal tubular portion, a first renal delivery member having a first distal port that is adapted to be delivered to a first delivery position within a first renal artery via a first corresponding renal ostium located at a first location along an abdominal aorta wall of an abdominal aorta in the patient, and a second renal delivery member having a second distal port that is adapted to be delivered to a second delivery position within a second renal artery via a second corresponding renal ostium located at a second location along the abdominal aorta wall that is different than the first location. Further, the infusion catheter can include a bifurcation joining the distal bifurcated portion and non-bifurcated proximal tubular portion of the catheter that provides for fluid communication between the first and second renal delivery members and the catheter inner lumen. The infusion catheter can also include a proximal coupler assembly that is adapted to be located externally of the patient when the first and second distal ports are positioned at the first and second delivery positions, respectively. The proximal coupler assembly can be coupled with the catheter inner lumen so that material can be delivered from outside the patient's body via the proximal coupler assembly, through the catheter inner lumen, through the first and second renal delivery members, through the first and second distal ports at the first and second delivery positions, respectively, and into the first and second renal arteries, also respectively. Hence, fluid agent can be delivered via the proximal coupler to each of the first and second distal ports within first and second renal arteries.

The system can be configured to deliver a treatment protocol to the patient based on the stage or severity of acute renal injury in the patient, the cause of acute renal injury in the patient, or both. For example, the system can be configured to deliver a vasodilator, an antioxidant, or both, when the acute kidney injury is characterized by an increase in serum creatinine by at least 50% over baseline, an absolute increase in serum creatinine of at least 0.3 mg/dL over baseline, a reduction in glomerular filtration rate of at least 25% compared to baseline, a decrease in urine output to 0.5 ml per kilogram of body weight or less per hour persisting for at least 6 hours, or any combination thereof. Relatedly, the system can be configured to deliver a vasodilator, an antioxidant, an anti inflammatory agent, an antibiotic agent, or any combination thereof, when the acute kidney injury is characterized by an increase in serum creatinine by at least 100% over baseline, a reduction in glomerular filtration rate of at least 50% compared to baseline, a decrease in urine output to 0.5 ml per kilogram of body weight or less per hour persisting for at least 12 hours, or any combination thereof. Similarly, a system can be configured to deliver a vasodilator, an antioxidant, an anti inflammatory agent, an antibiotic, a neurohormonally active agent, or any combination thereof, when the acute kidney injury is characterized by an increase in serum creatinine by at least 200% over baseline, an absolute serum creatinine rise of at least 0.5 mg/dL to a value of at least 4 mg/dL, a reduction in glomerular filtration rate of at least 75% compared to baseline, a decrease in urine output to 0.3 ml per kilogram of body weight or less per hour persisting for at least 24 hours, a decrease in total urine output over 12 hours to 200 ml or less, or any combination thereof. Likewise, a system can be configured to deliver vasodilator, an antioxidant, an anti inflammatory agent, a cytokine, a neurohormonally-active agent, or any combination thereof, when the acute kidney injury is caused at least in part by a kidney tumor or cancer. The tumor or cancer may include a renal cell carcinoma.

In another method aspect, embodiments of the present invention encompass techniques for treating a patient suffering from acute kidney injury that include placing a bifurcated renal artery infusion catheter within the abdominal aorta of the patient, where the bifurcated infusion catheter has a first renal delivery member with a first port and a second renal delivery member with a second port, placing the first renal delivery member within a first renal artery of the patient and placing the second renal delivery member within a second renal artery of the patient, and delivering an amount of a therapeutic agent from a therapeutic agent source through the bifurcated renal artery infusion catheter and into the first and second renal arteries via the first and second ports, respectively. The acute kidney injury can be characterized by an absolute increase in serum creatinine of more than 0.3 mg/dl within 48 hours, an increase in serum creatinine by at least 50% over baseline within 48 hours, a decrease in urine output to 0.5 ml per kilogram of body weight or less per hour persisting for at least 6 hours, or any combination thereof.

In still a further aspect, embodiments of the present invention encompass a system and method for treating a patient suffering from acute kidney injury. The acute kidney injury can be defined as one or more of an increase in serum creatinine by at least 50% over baseline, a reduction in glomerular filtration rate of at least 25% compared to baseline, or a decrease in urine output to 0.5 ml per kilogram of body weight or less per hour persisting for at least 6 hours. The system can include a bifurcated renal artery infusion catheter placed via percutaneous means. The bifurcated catheter can include a distal bifurcated portion and a proximal tubular portion, a catheter lumen traversing the length of the non-bifurcated proximal tubular portion of the catheter's length, a first renal delivery member with a first distal port that is adapted to be delivered to a first delivery position within a first renal artery via a first corresponding renal ostium located at a first location along an abdominal aorta wall of an abdominal aorta in a patient, a second renal delivery member with a second distal port that is adapted to be delivered to a second delivery position within a second renal artery via a second corresponding renal ostium located at a second location along the abdominal aorta wall that is different than the first location, a bifurcation joining the distal bifurcated portion and proximal tubular portion of the catheter and providing for fluid communication between the two renal delivery members and the catheter's inner lumen, and a proximal coupler assembly that is adapted to be located externally of the patient when the first and second distal ports are positioned at the first and second delivery positions, respectively. The proximal coupler assembly can be coupled to the catheter's inner lumen so as to deliver material from outside the patient's body via the proximal coupler assembly, through the catheter inner lumen, through the first and second renal delivery members, and into the first and second distal ports at the first and second delivery positions, respectively, and into the first and second renal arteries, also respectively. The system can also include a therapeutic agent source located externally to the patient. The therapeutic agent can include a vasodilator, an antioxidant, or both. The system can also include a means of delivering the therapeutic agent through the renal artery catheter via its proximal coupler to each of the first and second distal ports substantially within first and second renal arteries simultaneously or substantially simultaneously. In some cases, the kidney injury is caused by a toxic agent. The toxic agent can be radiocontrast media or a chemotherapy agent, for example. In some cases, the kidney injury is pre-renal in nature. For example, a pre-renal kidney injury can be caused by reduced cardiac output, reducing overall blood flow to the kidneys. In some cases, a pre-renal kidney injury is caused by systemic hypotension. Systemic hypotension can be caused by cardiorenal syndrome or acute decompensated heart failure. In some cases, a pre-renal kidney injury can be caused by a reduction in circulating volume due to hemorrhage. Similarly, the pre-renal kidney injury may have occurred post surgery. Optionally, the pre-renal kidney injury may have been caused by a reduction in local renal blood flow caused by hepatorenal syndrome. In some cases, the vasodilator is fenoldopam mesylate or an analog or derivative thereof, or dopamine or an analog or derivative thereof. Optionally, the vasodilator is a prostaglandin or analog or derivative thereof. In some cases, the antioxidant is sodium bicarbonate. Similarly, the antioxidant can be acetylcysteine. An intra-renally delivered agent can be dosed according to the patient's weight. Optionally, the intra-renally delivered agent can be administered at a fixed infusion rate regardless of the patient's weight. In some cases, the means of delivering the agent through the catheter is an infusion pump.

In another aspect, embodiments of the present invention include a system and method for treating a patient suffering from acute kidney injury, where the kidney injury can be defined as one or more of an increase in serum creatinine by at least 100% over baseline, a reduction in glomerular filtration rate of at least 50% compared to baseline, or a decrease in urine output to 0.5 ml per kilogram of body weight or less per hour persisting for at least 12 hours. The system can include a bifurcated renal artery infusion catheter placed via percutaneous means. The bifurcated catheter can include a distal bifurcated portion and a proximal tubular portion, a catheter lumen traversing the length of the non-bifurcated proximal tubular portion of the catheter's length, a first renal delivery member with a first distal port that is adapted to be delivered to a first delivery position within a first renal artery via a first corresponding renal ostium located at a first location along an abdominal aorta wall of an abdominal aorta in a patient, a second renal delivery member with a second distal port that is adapted to be delivered to a second delivery position within a second renal artery via a second corresponding renal ostium located at a second location along the abdominal aorta wall that is different than the first location, a bifurcation joining the distal bifurcated portion and proximal tubular portion of the catheter and providing for fluid communication between the two renal delivery members and the catheter's inner lumen, and a proximal coupler assembly that is adapted to be located externally of the patient when the first and second distal ports are positioned at the first and second delivery positions, respectively. In some cases, the proximal coupler assembly is coupled to the catheter's inner lumen so as to deliver material from outside the patient's body via the proximal coupler assembly, through the catheter inner lumen, through the first and second renal delivery members, and into the first and second distal ports at the first and second delivery positions, respectively, and into the first and second renal arteries, also respectively. The system may also include a therapeutic agent source located externally to the patient. The therapeutic agent can include one or more of a vasodilator, an antioxidant, an anti-inflammatory agent, or an antibiotic. The system can also include a means of delivering said therapeutic agent through the renal artery catheter via its proximal coupler to each of the first and second distal ports substantially within first and second renal arteries simultaneously or substantially simultaneously. In some cases, the kidney injury is caused by a toxic agent. The toxic agent can be radiocontrast media or a chemotherapy agent, for example. In some cases, the kidney injury is pre-renal in nature. For example, a pre-renal kidney injury can be caused by reduced cardiac output, reducing overall blood flow to the kidneys. In some cases, a pre-renal kidney injury is caused by systemic hypotension. Systemic hypotension can be caused by cardiorenal syndrome or acute decompensated heart failure. In some cases, a pre-renal kidney injury can be caused by a reduction in circulating volume due to hemorrhage. Similarly, the pre-renal kidney injury may have occurred post surgery. Optionally, the pre-renal kidney injury may have been caused by a reduction in local renal blood flow caused by hepatorenal syndrome. In some cases, the vasodilator is fenoldopam mesylate or an analog or derivative thereof, or dopamine or an analog or derivative thereof. Optionally, the vasodilator is a prostaglandin or analog or derivative thereof. In some cases, the antioxidant is sodium bicarbonate. Similarly, the antioxidant can be acetylcysteine. The antibiotic agent can be bacteriocidal or bacteriostatic. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

In another aspect, embodiments of the present invention encompass a system and method for treating a patient suffering from acute kidney injury, where the kidney injury is defined as one or more of an increase in serum creatinine by at least 200% over baseline, an absolute serum creatinine rise of at least 0.5 mg/dL to a value of at least 4 mg/dL, a reduction in glomerular filtration rate of at least 75% compared to baseline, a decrease in urine output to 0.3 ml per kilogram of body weight or less per hour persisting for at least 24 hours, or a decrease in total urine output over 12 hours to 200 ml or less. The system can include a bifurcated renal artery infusion catheter placed via percutaneous means. The bifurcated catheter can include a distal bifurcated portion and a proximal tubular portion, a catheter lumen traversing the length of the non-bifurcated proximal tubular portion of the catheter's length, a first renal delivery member with a first distal port that is adapted to be delivered to a first delivery position within a first renal artery via a first corresponding renal ostium located at a first location along an abdominal aorta wall of an abdominal aorta in a patient, a second renal delivery member with a second distal port that is adapted to be delivered to a second delivery position within a second renal artery via a second corresponding renal ostium located at a second location along the abdominal aorta wall that is different than the first location, a bifurcation joining the distal bifurcated portion and proximal tubular portion of the catheter and providing for fluid communication between the two renal delivery members and the catheter's inner lumen, and a proximal coupler assembly that is adapted to be located externally of the patient when the first and second distal ports are positioned at the first and second delivery positions, respectively. The proximal coupler assembly can be coupled to the catheter's inner lumen so as to deliver material from outside the patient's body via the proximal coupler assembly, through the catheter inner lumen, through the first and second renal delivery members, and into the first and second distal ports at the first and second delivery positions, respectively, and into the first and second renal arteries, also respectively. The system may also include a therapeutic agent source located externally to the patient. The therapeutic agent can include one or more of a vasodilator, an antioxidant, an anti-inflammatory agent, a diuretic, an antibiotic, or a neurohormonally active agent. The system may also include a means of delivering the therapeutic agent through the renal artery catheter via its proximal coupler to each of the first and second distal ports substantially within first and second renal arteries simultaneously. In some cases, the kidney injury is caused by a toxic agent. The toxic agent can be radiocontrast media or a chemotherapy agent, for example. In some cases, the kidney injury is pre-renal in nature. For example, a pre-renal kidney injury can be caused by reduced cardiac output, reducing overall blood flow to the kidneys. In some cases, a pre-renal kidney injury is caused by systemic hypotension. Systemic hypotension can be caused by cardiorenal syndrome or acute decompensated heart failure. In some cases, a pre-renal kidney injury can be caused by a reduction in circulating volume due to hemorrhage. Similarly, the pre-renal kidney injury may have occurred post surgery. Optionally, the pre-renal kidney injury may have been caused by a reduction in local renal blood flow caused by hepatorenal syndrome. In some cases, the vasodilator is fenoldopam mesylate or an analog or derivative thereof, or dopamine or an analog or derivative thereof. Optionally, the vasodilator is a prostaglandin or analog or derivative thereof. In some cases, the antioxidant is sodium bicarbonate. Similarly, the antioxidant can be acetylcysteine. The diuretic can be, for example, a loop diuretic such as furosemide. The antibiotic agent can be bacteriocidal or bacteriostatic. In some cases, the neurohormonally active agent is a natriuretic peptide, or an analog or derivative thereof. The natriuretic peptide may be A-type natriuretic peptide, B-type natriuretic peptide, or C-type natriuretic peptide. Relatedly, the natriuretic peptide can be a synthetic or bio-engineered natriuretic peptide. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

In still a further aspect, embodiments of the present invention encompass a system and method for treating a patient suffering from acute kidney injury, where the kidney injury is caused by a kidney tumor or cancer. The system can include a bifurcated renal artery infusion catheter placed via percutaneous means. The bifurcated catheter can include a distal bifurcated portion and a proximal tubular portion, a catheter lumen traversing the length of the non-bifurcated proximal tubular portion of the catheter's length, a first renal delivery member with a first distal port that is adapted to be delivered to a first delivery position within a first renal artery via a first corresponding renal ostium located at a first location along an abdominal aorta wall of an abdominal aorta in a patient, a second renal delivery member with a second distal port that is adapted to be delivered to a second delivery position within a second renal artery via a second corresponding renal ostium located at a second location along the abdominal aorta wall that is different than the first location, a bifurcation joining the distal bifurcated portion and proximal tubular portion of the catheter and providing for fluid communication between the two renal delivery members and the catheter's inner lumen, and a proximal coupler assembly that is adapted to be located externally of the patient when the first and second distal ports are positioned at the first and second delivery positions, respectively. The proximal coupler assembly can be coupled to the catheter's inner lumen so as to deliver material from outside the patient's body via the proximal coupler assembly, through the catheter inner lumen, through the first and second renal delivery members, and into the first and second distal ports at the first and second delivery positions, respectively, and into the first and second renal arteries, also respectively. The system may also include a therapeutic agent source located externally to the patient. The therapeutic agent can include one or more of a vasodilator, an antioxidant, an anti-inflammatory agent, a cytokine, or a neurohormonally-active agent. Further, the system may include a means of delivering said therapeutic agent through said renal artery catheter via its proximal coupler to each of the first and second distal ports substantially within first and second renal arteries simultaneously or substantially simultaneously. The kidney tumor or cancer may be related to renal cell carcinoma. The vasodilator can be fenoldopam mesylate or an analog or derivative thereof, or dopamine or an analog or derivative thereof. In some cases, the vasodilator is a prostaglandin or analog or derivative thereof. The antioxidant can be, for example, sodium bicarbonate or acetylcysteine. In some cases, the cytokine can be a lymphokine, such as interleukin-2, or a genetically engineered or modified version thereof. In some cases, the neurohormonally active agent is a natriuretic peptide, or an analog or derivative thereof. The natriuretic peptide can be A-type natriuretic peptide, B-type natriuretic peptide, or C-type natriuretic peptide. The natriuretic peptide can be a synthetic or bio-engineered natriuretic peptide. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

In another aspect, embodiments of the present invention encompass a method for treating a patient suffering from acute kidney injury, where the kidney injury is defined as one or more of an increase in serum creatinine by at least 50% over baseline, a reduction in glomerular filtration rate of at least 25% compared to baseline, or a decrease in urine output to 0.5 ml per kilogram of body weight or less per hour persisting for at least 6 hours. The method can include providing a percutaneous bifurcated renal artery infusion catheter placed within first and second renal arteries of the patient, where renal artery catheter includes a proximal coupler located externally to the patient in fluid communication with first and second delivery ports. The method can also include providing a therapeutic agent source located externally to the patient, in fluid communication with the proximal coupler of the bifurcated renal artery infusion catheter, where the therapeutic agent includes one or more of a vasodilator or an antioxidant. Further, the method can include delivering the therapeutic agent through the renal artery catheter via its proximal coupler to each of the first and second distal ports substantially within first and second renal arteries simultaneously or substantially simultaneously. In some cases, the kidney injury is caused by a toxic agent. The toxic agent can be radiocontrast media or a chemotherapy agent, for example. In some cases, the kidney injury is pre-renal in nature. For example, a pre-renal kidney injury can be caused by reduced cardiac output, reducing overall blood flow to the kidneys. In some cases, a pre-renal kidney injury is caused by systemic hypotension. Systemic hypotension can be caused by cardiorenal syndrome or acute decompensated heart failure. In some cases, a pre-renal kidney injury can be caused by a reduction in circulating volume due to hemorrhage. Similarly, the pre-renal kidney injury may have occurred post surgery. Optionally, the pre-renal kidney injury may have been caused by a reduction in local renal blood flow caused by hepatorenal syndrome. In some cases, the vasodilator is fenoldopam mesylate or an analog or derivative thereof, or dopamine or an analog or derivative thereof. Optionally, the vasodilator is a prostaglandin or analog or derivative thereof. In some cases, the antioxidant is sodium bicarbonate or acetylcysteine. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

In another aspect, embodiments of the present invention provide a method for treating a patient suffering from acute kidney injury, where the kidney injury is defined as one or more of an increase in serum creatinine by at least 100% over baseline, a reduction in glomerular filtration rate of at least 50% compared to baseline, or a decrease in urine output to 0.5 ml per kilogram of body weight or less per hour persisting for at least 12 hours. The method can include providing a percutaneous bifurcated renal artery infusion catheter placed within first and second renal arteries of the patient, where the renal artery catheter includes a proximal coupler located externally to the patient in fluid communication with first and second delivery ports. The method can also include providing a therapeutic agent source located externally to the patient, in fluid communication with the proximal coupler of the bifurcated renal artery infusion catheter, where the therapeutic agent includes one or more of a vasodilator, an antioxidant, an anti-inflammatory agent, or an antibiotic. Further, the method may include delivering the therapeutic agent through the renal artery catheter via its proximal coupler to each of the first and second distal ports substantially within first and second renal arteries simultaneously.

In some cases, the kidney injury is caused by a toxic agent. The toxic agent can be radiocontrast media or a chemotherapy agent, for example. In some cases, the kidney injury is pre-renal in nature. For example, a pre-renal kidney injury can be caused by reduced cardiac output, reducing overall blood flow to the kidneys. In some cases, a pre-renal kidney injury is caused by systemic hypotension. Systemic hypotension can be caused by cardiorenal syndrome or acute decompensated heart failure. In some cases, a pre-renal kidney injury can be caused by a reduction in circulating volume due to hemorrhage. Similarly, the pre-renal kidney injury may have occurred post surgery. Optionally, the pre-renal kidney injury may have been caused by a reduction in local renal blood flow caused by hepatorenal syndrome. In some cases, the vasodilator is fenoldopam mesylate or an analog or derivative thereof, or dopamine or an analog or derivative thereof. Optionally, the vasodilator is a prostaglandin or analog or derivative thereof. In some cases, the antioxidant is sodium bicarbonate. Similarly, the antioxidant can be acetylcysteine. The antibiotic agent can be bacteriocidal or bacteriostatic. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

In another aspect, embodiments of the present invention encompass a method for treating a patient suffering from acute kidney injury, where the kidney injury is defined as one or more of an increase in serum creatinine by at least 200% over baseline, an absolute serum creatinine rise of at least 0.5 mg/dL to a value of at least 4 mg/dL, a reduction in glomerular filtration rate of at least 75% compared to baseline, a decrease in urine output to 0.3 ml per kilogram of body weight or less per hour persisting for at least 24 hours, or a decrease in total urine output over 12 hours to 200 ml or less. The method can include providing a percutaneous bifurcated renal artery infusion catheter placed within first and second renal arteries of the patient, where the renal artery catheter includes a proximal coupler located externally to the patient in fluid communication with first and second delivery ports. The method can also include providing a therapeutic agent source located externally to the patient, in fluid communication with the proximal coupler of the bifurcated renal artery infusion catheter, where the therapeutic agent includes one or more of a vasodilator, an antioxidant, an anti-inflammatory agent, an antibiotic, or a neurohormonally active agent. Further, the method can include delivering the therapeutic agent through the renal artery catheter via its proximal coupler to each of the first and second distal ports substantially within first and second renal arteries simultaneously or substantially simultaneously. In some cases, the kidney injury is caused by a toxic agent. The toxic agent can be radiocontrast media or a chemotherapy agent, for example. In some cases, the kidney injury is pre-renal in nature. For example, a pre-renal kidney injury can be caused by reduced cardiac output, reducing overall blood flow to the kidneys. In some cases, a pre-renal kidney injury is caused by systemic hypotension. Systemic hypotension can be caused by cardiorenal syndrome or acute decompensated heart failure. In some cases, a pre-renal kidney injury can be caused by a reduction in circulating volume due to hemorrhage. Similarly, the pre-renal kidney injury may have occurred post surgery. Optionally, the pre-renal kidney injury may have been caused by a reduction in local renal blood flow caused by hepatorenal syndrome. In some cases, the vasodilator is fenoldopam mesylate or an analog or derivative thereof, or dopamine or an analog or derivative thereof. Optionally, the vasodilator is a prostaglandin or analog or derivative thereof. In some cases, the antioxidant is sodium bicarbonate. Similarly, the antioxidant can be acetylcysteine. The diuretic can be, for example, a loop diuretic such as furosemide. The antibiotic agent can be bacteriocidal or bacteriostatic. In some cases, the neurohormonally active agent is a natriuretic peptide, or an analog or derivative thereof. The natriuretic peptide may be A-type natriuretic peptide, B-type natriuretic peptide, or C-type natriuretic peptide. Relatedly, the natriuretic peptide can be a synthetic or bio-engineered natriuretic peptide. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

In still a further aspect, embodiments of the present invention encompass a method for treating a patient suffering from acute kidney injury, where the kidney injury is caused by a kidney tumor or cancer. The method can include providing a percutaneous bifurcated renal artery infusion catheter placed within first and second renal arteries of the patient, where the renal artery catheter includes a proximal coupler located externally to the patient in fluid communication with first and second delivery ports. The method can also include providing a therapeutic agent source located externally to the patient, in fluid communication with the proximal coupler of the bifurcated renal artery infusion catheter, where the therapeutic agent includes one or more of a vasodilator, an antioxidant, an anti-inflammatory agent, a cytokine, or a neurohormonally-active agent. The method may also include delivering the therapeutic agent through the renal artery catheter via its proximal coupler to each of the first and second distal ports substantially within first and second renal arteries simultaneously or substantially simultaneously. The kidney tumor or cancer may be related to renal cell carcinoma. The vasodilator can be fenoldopam mesylate or an analog or derivative thereof, or dopamine or an analog or derivative thereof. In some cases, the vasodilator is a prostaglandin or analog or derivative thereof. The antioxidant can be, for example, sodium bicarbonate or acetylcysteine. In some cases, the cytokine can be a lymphokine, such as interleukin-2, or a genetically engineered or modified version thereof. In some cases, the neurohormonally active agent is a natriuretic peptide, or an analog or derivative thereof. The natriuretic peptide can be A-type natriuretic peptide, B-type natriuretic peptide, or C-type natriuretic peptide. The natriuretic peptide can be a synthetic or bio-engineered natriuretic peptide. An intra-renally delivered agent can be dosed according to the patient's weight. Optionally, the intra-renally delivered agent can be administered at a fixed infusion rate regardless of the patient's weight. The agent can also be delivered at a variable infusion rate independent of the patient's weight. In some cases, the means of delivering the agent through the catheter is an infusion pump.

Some alternative embodiments of the present invention encompass techniques involve directing an active agent or fluid to a renal system in a patient. Such approaches may include positioning a renal device at a location within the patient's aorta that is adjacent to an ostium of at least one renal artery extending from the aorta, and segregating or dividing a first portion of aortic flow and a second portion of aortic flow from an aortic blood flow within the aorta with the renal device. Accordingly, these embodiments may include techniques discussed in, for example, U.S. Patent Publication No. 2004/0064091, the entire contents of which are hereby incorporated by reference. The technique can also include directing the first portion of aortic blood flow into the at least one renal artery, while allowing the second portion to flow across the location and downstream of the ostium. Further, the technique may include locally delivering a volume of fluid having a diagnostic, prophylactic or therapeutic agent to the at least one renal artery in the patient with the renal device. Optionally, the technique may involve positioning a medical device within an aorta of the patient upstream from an ostium of the at least one renal artery along the aorta of the patient, while locally delivering the volume of fluid to the at least one renal artery with the renal device.

For a fuller understanding of the nature and advantages of the present invention, reference should be had to the ensuing detailed description taken in conjunction with the accompanying drawings.

### BRIEF DESCRIPTION OF THE DRAWINGS

FIG. 1 illustrates aspects of diagnostic and therapeutic methods according to embodiments of the present invention.

FIG. 2 illustrates aspects of a renal treatment system according to embodiments of the present invention.

### DETAILED DESCRIPTION OF THE INVENTION

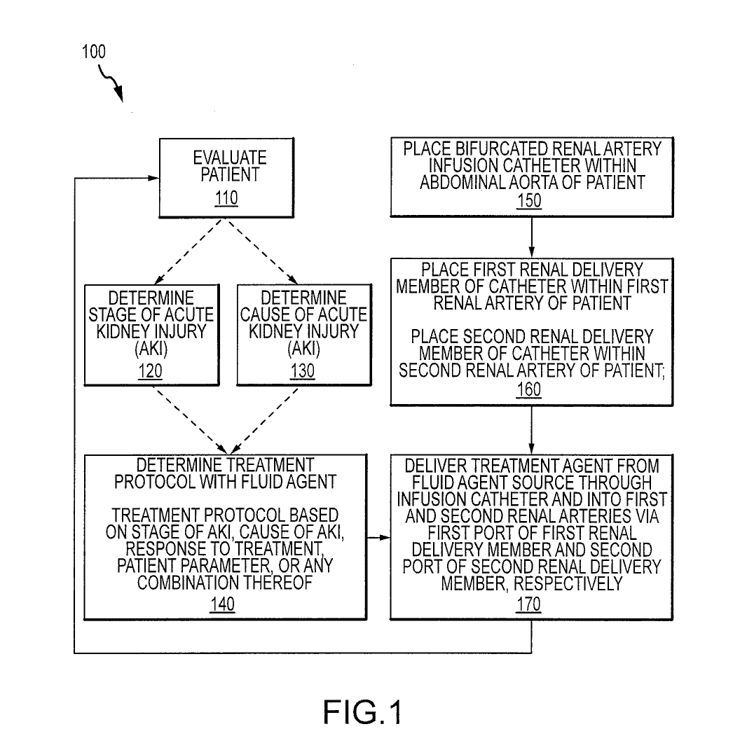

Embodiments of the present invention encompass systems and methods for diagnosing and treating patients who are at risk of developing or are suffering from acute kidney injury. Turning now to the drawings, FIG. 1 illustrates aspects of a renal diagnosis and treatment technique 100. An exemplary method may include the step of evaluating the patient 110. Evaluation can be based on any of a variety of clinical criteria. For example, a patient can be evaluated by a physician or other laboratory or medical personnel on the basis of serum creatinine level, glomerular filtration rate, urine output, and the like. An evaluation can also take into consideration certain patient parameters, such as age, weight, sex, medical history, and the like. Method 100 may also include the step of determining the stage or extent of an acute kidney injury 120 in the patient. The clinical stage of an acute kidney injury can be determine based on an analysis of clinical criteria observed or detected as part of a patient evaluation. In some cases, a clinical stage of AKI can reflect the level of kidney function in the patient. For example, in some cases a mild stage of acute kidney injury can be characterized by an increase in serum creatinine by at least 50% over baseline, an absolute increase in serum creatinine of at least 0.3 mg/dL over baseline, a reduction in glomerular filtration rate of at least 25% compared to baseline, a decrease in urine output to 0.5 ml per kilogram of body weight or less per hour persisting for at least 6 hours, or any combination thereof. Method 100 can also include the step of determining the cause of an acute kidney injury 130. For example, a physician or other laboratory or medical personnel may determine that the patient suffers from acute kidney injury due to administration of a radiocontrast agent. Method 100 can further include the step of determining a treatment protocol 140 for the patient. The treatment protocol typically involves administration of a fluid agent to the patient, and is determined or selected based on the stage of AKI, the cause of AKI, or both. For example, if the patient is determined to exhibit a mild stage of AKI, the treatment protocol may include administering a fluid agent that may include a vasodilator, an antioxidant, or both, to the patient. Optionally, a treatment protocol can be designed or selected based on a patient parameter, such as weight, age, sex, medical history, and the like. In some cases, a treatment protocol can be designed based on a patient's response to a previous or currently administered treatment regimen.

Typically, the fluid agent is administered directly to the renal arteries of the patient. To provide such a treatment, method 100 may include the step of placing a bifurcated renal artery infusion catheter within the abdominal aorta of the patient 150, where the bifurcated infusion catheter has a first renal delivery member with a first port and a second renal delivery member with a second port. Method 100 can also include the step of placing renal delivery members within renal arteries of the patient 160. For example, the method can include placing a first renal delivery member of the infusion catheter within a first renal artery of the patient, and placing a second renal delivery member of the infusion catheter within a second renal artery of the patient. Method 100 can also include the step of administering or delivering fluid agent directly to the renal arteries 170. For example, the administration step can include delivering a treatment agent from a fluid agent source through the infusion catheter and into the first and second renal arteries via the first port of the first renal delivery member and the second port of the second renal delivery member, respectively. Optionally, method steps may be performed in an iterative fashion. For example, a method may include an evaluation step, followed by a treatment administration step, and this process can be repeated as needed or desired.

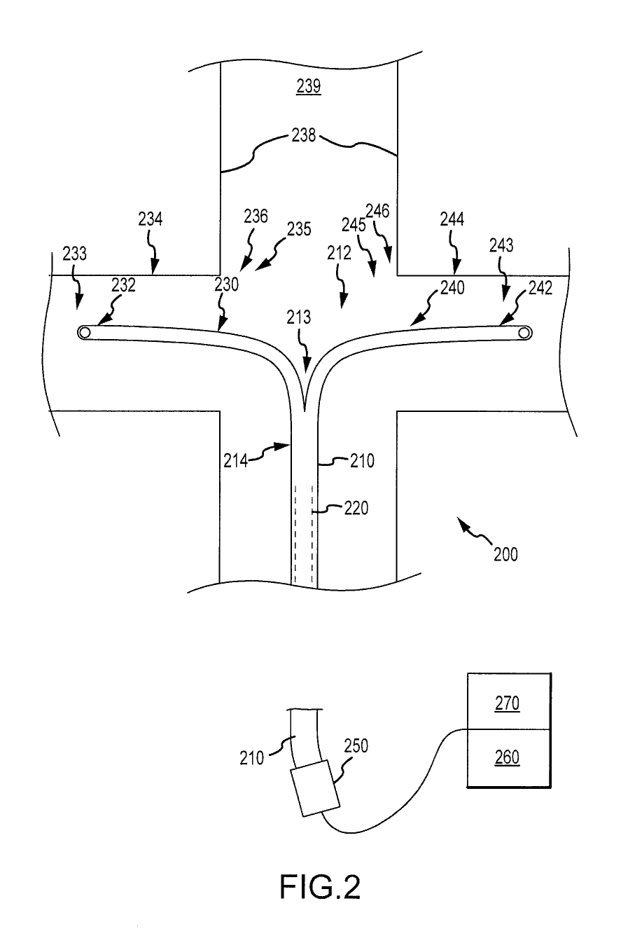

FIG. 2 depicts aspects of a system for treating a patient who may be suffering from acute kidney injury. System 200 includes a bifurcated renal artery infusion catheter 210 having a distal bifurcated portion 212 and a non-bifurcated proximal tubular portion 214. Catheter 210 also includes a catheter inner lumen 220 traversing a length of the non-bifurcated proximal tubular portion, a first renal delivery member 230 having a first distal port 232, and a second renal delivery member 240 having a second distal port 242. First distal port 232 is adapted to be delivered to a first delivery position 233 within a first renal artery 234 via a first corresponding renal ostium 235 located at a first location 236 along an abdominal aorta wall 238 of an abdominal aorta 239 in the patient. Second distal port 242 is adapted to be delivered to a second delivery position 243 within a second renal artery 244 via a second corresponding renal ostium 245 located at a second location 246 along the abdominal aorta wall 238 that is different than the first location 236. Catheter 210 also includes a bifurcation 213 that joins the distal bifurcated portion 212 and non-bifurcated proximal tubular portion 214 and provides for fluid communication between the first and second renal delivery members 230, 240 and the catheter inner lumen 220. Catheter 210 can also include a proximal coupler assembly 250 that is adapted to be located externally of the patient when the first and second distal ports 232, 242 are positioned at the first and second delivery positions 233, 243, respectively. Proximal coupler assembly 250 can be coupled with or in fluid communication with catheter inner lumen 220 so that material such as fluid can be delivered from outside the patient's body via proximal coupler assembly 250, through catheter inner lumen 220, through the first and second renal delivery members 230, 240, through the first and second distal ports 232, 242 at the first and second delivery positions 233, 243, respectively, and into the first and second renal arteries 234, 244, also respectively. System 200 can also include a therapeutic agent source 260 or other fluid or material source which may be located externally to the patient. System 200 can also include a mechanism 270 that delivers therapeutic agent through the bifurcated renal artery catheter 210 via the proximal coupler 250 to each of the first and second distal ports 232, 242 within first and second renal arteries 234, 244. Mechanism 270 can include, for example, an infusion pump.

An exemplary method may involve inserting one or more portions of system 200 through a minimally invasive incision in a patient, and into or toward a descending aorta, such as a thoracic aorta or abdominal aorta of the patient. The operator may advance portions of system 200 toward first and second renal arteries 234, 244. The operator can deploy first delivery member or branch 230 of bifurcated renal catheter system 200 into first renal artery 234. The operator can also deploy second delivery member or catheter branch 240 of bifurcated renal catheter system 200 into second renal artery 244. Fluid delivery can be performed as an adjunct to a surgical intervention or medical procedure.

Targeted Renal Therapy (TRT), or the direct delivery of therapeutic agents to the kidneys via the renal arteries, offers a way to increase the therapeutic windows of certain drugs that may have a beneficial renal effect in AKI. The benefits of TRT include the following:
(a) Delivery of agents directly to the kidneys via the renal arteries increases the local dose of the agent over that which could be achieved via systemic (intravenous, IV) delivery, as the dilution by the blood is significantly less, and
(b) Delivery directly to the kidneys can take advantage of renal first-pass, in which the kidney can clear a substantial portion of the drug immediately into the urine before its return to the systemic circulation via the renal veins; thus, systemic exposure is limited.

TRT may be provided via a series of bifurcated renal artery infusion catheter and sheath systems as previously disclosed in, for example, U.S. Pat. Nos. 6,994,700 and 7,104,981 and published U.S. patent application Ser. Nos. 11/084,738 and 11/129,101, all commonly owned and incorporated herein by reference. These percutaneous catheter systems provide for rapid and facile access to both renal arteries of a patient, in many cases simultaneously or substantially simultaneously, thus allowing for the delivery of TRT.

TRT can be applied to patients with various agents in many clinical settings to mitigate acute kidney injury. For example, TRT can be applied with fenoldopam mesylate, a dopamine D₁-like receptor agonist vasodilator. Such treatment can increase glomerular filtration rate (GFR) significantly above baseline (approximately 25%) and significantly more than IV administration, with no significant effect on blood pressure, in patients with moderate to severe underlying renal dysfunction. The increase in GFR can be seen in the setting of an insult to the kidneys in the form of a nephrotoxic radio-contrast agent. In similar patients receiving the same contrast agent insult who do not receive TRT, a substantial decline in GFR can be seen (approximately 14%). (Teirstein P., et al, Am J Cardiol 97:1076-81 (2006)).

Studies in the literature have demonstrated potential positive effects of IV fenoldopam in the setting of AKI. For example, Morelli et al demonstrated improved outcomes in preventing kidney failure in septic patients. However, this study required the infusion of low-dose fenoldopam (0.09 mcg/kg/min, to prevent hypotension) over a protracted course (average of one week or more of drug infusion in the ICU) (Morelli, A. et al., Crit Care Med 33:2451-6 (2005)).

TRT with fenoldopam mesylate or dopamine can provide an effective treatment option for pre-renal AKI patients. Embodiments of the present invention encompass the use of TRT to treat AKI and facilitate dialysis removal (Allie, D. et al., J Invasive Cardiol 19(2):E27-30 (2007)). TRT can be used with fenoldopam or dopamine, as well as other agents that have favorable renal function and renal first-pass effects. Local renal artery infusion of dopamine has been shown to increase global renal blood flow in patients with renal disease (Manoharan G. et al, J Am Coll Cardiol 47(3):620-5 (2005)). Other possible agents include, for example, members of the prostaglandin family, antioxidant and pH-modifying agents, and recombinant human peptides/proteins such as natriuretic peptides and certain cytokines.

Embodiments of the present invention encompass means for determining the relative kidney health of a patient. These include blood lab measurements of various substances such as creatinine (a by-product of muscle cell activity) and neutrophil gelatinase-associated lipocalin (NGAL, an early marker of kidney response to ischemic injury). Baseline values for patients can establish a risk of developing AKI, and attainment of threshold values or comparisons of sequential measurements at various time points after a potential renal injury can demonstrate the likelihood and extent of any renal injury that may have occurred. Thus, there are means to determine that appropriate time for intervention with TRT in order to prevent or treat AKI in certain patients. This is aided by the knowledge of the timing of the renal insult and disease progression in the case of hospital-acquired AKI.

Embodiments of the present invention encompass catheter systems and methods such as those previously described in U.S. Pat. Nos. 6,994,700 and 7,104,981 and published U.S. patent application Ser. Nos. 11/084,738, 11/129,101, and 11/768,390 (as previously noted and incorporated herein). These devices and methods incorporate bifurcated renal artery catheters designed and constructed so as to enable the physician user to easily and quickly access both renal arteries simultaneously with a single catheter device, often without the need for ancillary guide wires or catheters, and through a single vascular access site. This bilateral renal artery access then provides a drug delivery pathway directly to both of the patient's kidneys, allowing for administration of TRT as previously described.

The bilateral devices may be used or modified for use in the ICU setting, where many patients susceptible to or suffering from AKI or impending AKI might reside in the hospital. Relatedly, embodiments of the present invention encompass features or adaptations such as the ability to be guided into position without fluoroscopy, a heightened ability to remain stable in the body for longer periods of TRT without direct physician monitoring, and the like. Some of these features (e.g., means for renal positional stability) are described in particular in previously-disclosed co-owned U.S. patent application Ser. No. 11/129,101.

In some embodiments, a method aspect revolves around specific drug treatment as delivered via TRT for the specific application of treating patients with impending or already-developed AKI. Depending on the patient's condition, AKI etiology, and stage of disease (per, for example, the previously-discussed RIFLE or AKIN criteria), drug treatment may be tailored to specifically address the patient's condition and provide for an effective or optimized treatment in terms of safety and efficacy.

For example, in the case of pre-renal injury due to a reduction in renal blood flow, which may be caused by systemic hypotension, cardiorenal syndrome, hepatorenal syndrome, and the like, it is possible to administer with TRT agents such as renal vasodilators, antioxidants, and neurohormonal regulatory agents to improve blood flow, reduce secondary injury due to reactive oxygen species (ROS), and provide for suppression of hormones that may be causing an imbalance in the kidneys' natriuresis and diuresis. Local administration of these agents via TRT can prevent or ameliorate the systemic hypotension that often results with IV delivery, which in turn would likely exacerbate the renal blood flow problems.

As a further example, in the case of a post-renal kidney injury as may be secondary to renal vein thrombosis, TRT with anticoagulation agents can be administered, for example in conjunction with antioxidants to preclude or inhibit ROS injury as noted above. By providing the anticoagulation locally, systemic or remote hemorrhage becomes less likely.

As yet another example, in the case of sepsis, anti-inflammatory agents and antibiotics can be administered via TRT along with renal vasodilators to allow the kidney to rid the bloodstream of infectious wastes without itself becoming unduly compromised. Again, local administration of the vasodilator prevents or inhibits systemic hypotension that could worsen the clinical situation as it relates to renal function, while local anti-inflammatory delivery can allow preservation of kidney function without compromising the rest of the body's natural ability to fight the underlying infection. Local antibiotics can be used in conjunction with systemic doses in the setting of sepsis.

As yet another example, in cases of direct renal toxicity from non-renal causes, such as the systemic or remote administration of radiocontrast agents for vascular or other imaging or chemotherapy agents for various cancer conditions, where the kidney suffers injury from the toxic agents as it attempts to clear them from the circulation, TRT with vasodilators and antioxidants can provide benefit by allowing the kidney to filter the material more quickly (due to increases in renal blood flow and GFR) and by preventing or inhibiting secondary ROS injury.

As yet another example, in the case of renal dysfunction caused by renal cell carcinoma (RCC), patients are often treated with repeated bolus IV infusions of interleukin-2 to stimulate immune response that can attack tumor cells. However, the administration of therapeutically advantageous doses of interleukin-2 has been associated with major adverse events, most notably hypotension. The pharmacokinetic characteristics of interleukin-2 are favorable for TRT, in that it is metabolized and cleared within the kidney by both glomerular filtration and peritubular extraction (PROLEUKIN® Aldesleukin for Injection. Package Insert & Prescribing Information. Chiron Corporation (2000)). As discussed previously, hypotension is a major cause for pre-renal AKI, and thus the ability to avoid hypotension in treatment of the underlying disease (in this case RCC) is desirable. Thus, interleukin-2 delivered via TRT is a more attractive treatment option than is systemic IV delivery for patients suffering from RCC.

Likewise, the TRT treatment may be determined according to the stage or severity of the AKI progression. As an example, in early stages of kidney injury, when the decline in renal function is due primarily to acute reductions in renal blood flow, TRT treatment with vasodilators and/or antioxidants can provide relief from dysfunction as this can correct or improve the blood flow imbalance and prevent or inhibit further ROS-induced cellular injury. However, should kidney injury persist prior to treatment being available, additional agents such as anti-inflammatory agents may be employed to counteract injury response mechanisms within the kidney that may further exacerbate the decline in renal function. As well, neurohormonal agents can provide benefit in later-stage progression as the rest of the body begins to respond to the kidneys' decline.

As noted above, method embodiments may include monitoring patients prior to and in response to protocols for treating or preventing acute kidney injury. Hence, methods may include administering to the patient a dosage regime of a treatment agent directly to the renal arteries, and monitoring the condition of the patient responsive to the treatment, wherein the monitoring indicates whether any change has occurred in renal function, and the extent of such change. For example, monitoring may involve evaluating clinical indicators of renal function such as serum creatinine or urine output of the patient. Methods may also include determining whether to change a treatment regimen based on the observed or detected responsive changes associated with renal function. Further, methods may include any number of monitoring and evaluating steps, and administration of treatment protocols, optionally adjusted based on the monitoring and evaluation steps, until there is a clinical indication of improvement in renal function or a reduction in the severity of acute kidney injury, or until there is a contraindication to further treatment.

Embodiments may also include methods for treating a patient suffering from acute kidney injury based on a tiered classification of renal function, optionally depending on the stage of acute kidney injury, the cause of the acute kidney injury, or both. For example, a method can include placing a bifurcated renal artery infusion catheter within the abdominal aorta of the patient, where the bifurcated infusion catheter has a first renal delivery member with a first port and a second renal delivery member with a second port, placing the first renal delivery member within a first renal artery of the patient, placing the second renal delivery member within a second renal artery of the patient. A method can also include delivering an amount of a therapeutic agent from a therapeutic agent source through the bifurcated renal artery infusion catheter and into the first and second renal arteries via the first and second ports, respectively, where the therapeutic agent is selected based on the stage or severity of the acute kidney injury, the cause of the acute kidney injury, or both.

The referenced patents, patent applications, and scientific literature, including accession numbers to GenBank database sequences, referred to herein are hereby incorporated by reference in their entirety. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

While the above provides a full and complete disclosure of certain embodiments of the present invention, various modifications, alternate constructions and equivalents may be employed as desired. Therefore, the above description and illustrations should not be construed as limiting the invention, which is defined by the appended claims.

What is claimed is:

1. A method for treating a patient suffering from acute kidney injury, comprising:
placing a bifurcated renal artery infusion catheter within the abdominal aorta of the patient, the bifurcated infusion catheter having a first renal delivery member with a first port and a second renal delivery member with a second port;
placing the first renal delivery member within a first renal artery of the patient, and placing the second renal delivery member within a second renal artery of the patient; and
delivering an amount of a therapeutic agent from a therapeutic agent source through the bifurcated renal artery infusion catheter and into the first and second renal arteries via the first and second ports, respectively,
wherein the acute kidney injury is characterized by at least one condition selected from the group consisting of an increase in serum creatinine by at least 50% over baseline, an absolute increase in serum creatinine of at least 0.3 mg/dL over baseline, a reduction in glomerular filtration rate of at least 25% compared to baseline, and a decrease in urine output to 0.5 ml per kilogram of body weight or less per hour persisting for at least 6 hours.

2. A method as in claim 1, wherein the therapeutic agent comprises a member selected from the group consisting of a vasodilator and an antioxidant.

3. A method as in claim 1, wherein the kidney injury was caused by a toxic agent selected from the group consisting of a radiocontrast media, a non-steroidal anti-inflammatory drug (NSAID), and a chemotherapy agent.

4. A method as in claim 1, wherein acute kidney injury comprises a pre renal kidney injury caused by a member selected from the group consisting of a reduced cardiac output leading to reduced overall blood flow to the kidneys, trauma, reduced blood oxygenation, systemic toxicity caused by reaction to injury in another organ, systemic hypotension resulting from cardiorenal syndrome or acute decompensated heart failure, a reduction in circulating volume due to hemorrhage, a surgical procedure, and a reduction in local renal blood flow resulting from hepatorenal syndrome.

5. A system as in claim 1, wherein the vasodilator comprises a member selected from the group consisting of fenoldopam mesylate or an analog or derivative thereof, dopamine or an analog or derivative thereof, and a prostaglandin or analog or derivative thereof, and wherein the antioxidant comprises a member selected from the group consisting of ascorbic acid, sodium bicarbonate, and acetylcysteine.

6. A method as in claim 1, wherein the acute kidney injury is characterized by at least one condition selected from the group consisting of an increase in serum creatinine by at least 100% over baseline, a reduction in glomerular filtration rate of at least 50% compared to baseline, and a decrease in urine output to 0.5 ml per kilogram of body weight or less per hour persisting for at least 12 hours.

7. A method as in claim 6, wherein the therapeutic agent comprises a member selected from the group consisting of a vasodilator, an antioxidant, an anti inflammatory agent, and an antibiotic agent.

8. A method as in claim 7, wherein the vasodilator comprises a member selected from the group consisting of fenoldopam mesylate or an analog or derivative thereof, dopamine or an analog or derivative thereof, and a prostaglandin or analog or derivative thereof, wherein the antioxidant comprises a member selected from the group consisting of ascorbic acid, sodium bicarbonate, and acetylcysteine, wherein the anti inflammatory agent comprises a P-38 kinase inhibitor, and wherein the antibiotic comprises a member selected from the group consisting of a bactericidal agent and a bacteriostatic agent.

9. A method as in claim 1, wherein the acute kidney injury is characterized by at least one condition selected from the group consisting of an increase in serum creatinine by at least 200% over baseline, an absolute serum creatinine rise of at least 0.5 mg/dL to a value of at least 4 mg/dL, a reduction in glomerular filtration rate of at least 75% compared to baseline, a decrease in urine output to 0.3 ml per kilogram of body weight or less per hour persisting for at least 24 hours, and a decrease in total urine output over 12 hours to 200 ml or less.

10. A method as in claim 9, wherein the therapeutic agent comprises a member selected from the group consisting of a vasodilator, an antioxidant, a diuretic, an anti inflammatory agent, an antibiotic, and a neurohormonally active agent.

11. A method as in claim 10, wherein the vasodilator comprises a member selected from the group consisting of fenoldopam mesylate or an analog or derivative thereof, dopamine or an analog or derivative thereof, and a prostaglandin or analog or derivative thereof, wherein the antioxidant comprises a member selected from the group consisting of ascorbic acid, sodium bicarbonate, and acetylcysteine, wherein the diuretic comprises hydrochlorothiazide (HCTZ), spironolactone, and a loop diuretic, wherein the antibiotic comprises a member selected from the group consisting of a bactericidal agent and a bacteriostatic agent, and wherein the neurohormonally active agent comprises a natriuretic peptide or an analog or derivative thereof.

12. A method as in claim 11, wherein the loop diuretic comprises furosemide, and wherein the natriuretic peptide comprises a member selected from the group consisting of A-type natriuretic peptide, B-type natriuretic peptide, C-type natriuretic peptide, a synthetic natriuretic peptide, and a bio-engineered natriuretic peptide.

13. A method as in claim 1, wherein the acute kidney injury is caused at least in part by a kidney tumor or cancer comprising a renal cell carcinoma, and the therapeutic agent comprises a member selected from the group consisting of a vasodilator, an antioxidant, an anti inflammatory agent, a cytokine, and a neurohormonally-active agent.

14. A method as in claim 13, wherein the vasodilator comprises a member selected from the group consisting of fenoldopam mesylate or an analog or derivative thereof, dopamine or an analog or derivative thereof, and a prostaglandin or analog or derivative thereof, wherein the antioxidant comprises a member selected from the group consisting of sodium bicarbonate and acetylcysteine, wherein the cytokine comprises a lymphokine, and wherein the neurohormonally active agent comprises a natriuretic peptide or an analog or derivative thereof.

15. A method as in claim 14, wherein the lymphokine comprises interleukin-2 or a genetically engineered or modified version thereof, and wherein the natriuretic peptide comprises a member selected from the group consisting of A-type natriuretic peptide, B-type natriuretic peptide, C-type natriuretic peptide, a synthetic natriuretic peptide, and a bio-engineered natriuretic peptide.

16. A system for treating a patient suffering from acute kidney injury, comprising:
a. a bifurcated renal artery infusion catheter placed via a percutaneous means, the bifurcated catheter comprising:
i. a distal bifurcated portion and a non-bifurcated proximal tubular portion;
ii. a catheter inner lumen traversing a length of the non-bifurcated proximal tubular portion of the infusion catheter;
iii. a first renal delivery member having a first distal port that is adapted to be delivered to a first delivery position within a first renal artery via a first corresponding renal ostium located at a first location along an abdominal aorta wall of an abdominal aorta in the patient;
iv. a second renal delivery member having a second distal port that is adapted to be delivered to a second delivery position within a second renal artery via a second corresponding renal ostium located at a second location along the abdominal aorta wall that is different than the first location;
v. a bifurcation joining the distal bifurcated portion and non-bifurcated proximal tubular portion of the catheter and providing for fluid communication between the first and second renal delivery members and the catheter inner lumen; and
vi. a proximal coupler assembly that is adapted to be located externally of the patient when the first and second distal ports are positioned at the first and second delivery positions, respectively, wherein the proximal coupler assembly is coupled with the catheter inner lumen so that material can be delivered from outside the patient's body via the proximal coupler assembly, through the catheter inner lumen, through the first and second renal delivery members, through the first and second distal ports at the first and second delivery positions, respectively, and into the first and second renal arteries, also respectively;
b. a therapeutic agent source located externally to the patient; and
c. a mechanism that delivers the therapeutic agent through the bifurcated renal artery catheter via the proximal coupler to each of the first and second distal ports within first and second renal arteries,
wherein the acute kidney injury is characterized by at least one condition selected from the group consisting of an increase in serum creatinine by at least 50% over baseline, an absolute increase in serum creatinine of at least 0.3 mg/dL over baseline, a reduction in glomerular filtration rate of at least 25% compared to baseline, and a decrease in urine output to 0.5 ml per kilogram of body weight or less per hour persisting for at least 6 hours.

17. A system as in claim 16, wherein the therapeutic agent comprises a member selected from the group consisting of a vasodilator and an antioxidant.

18. A system as in claim 16, wherein the acute kidney injury is characterized by at least one condition selected from the group consisting of an increase in serum creatinine by at least 100% over baseline, a reduction in glomerular filtration rate of at least 50% compared to baseline, and a decrease in urine output to 0.5 ml per kilogram of body weight or less per hour persisting for at least 12 hours, and wherein the therapeutic agent comprises a member selected from the group consisting of a vasodilator, an antioxidant, an anti inflammatory agent, and an antibiotic agent.

19. A system as in claim 16, wherein the acute kidney injury is characterized by at least one condition selected from the group consisting of an increase in serum creatinine by at least 200% over baseline, an absolute serum creatinine rise of at least 0.5 mg/dL to a value of at least 4 mg/dL, a reduction in glomerular filtration rate of at least 75% compared to baseline, a decrease in urine output to 0.3 ml per kilogram of body weight or less per hour persisting for at least 24 hours, and a decrease in total urine output over 12 hours to 200 ml or less, and wherein the therapeutic agent comprises a member selected from the group consisting of a vasodilator, an antioxidant, an anti inflammatory agent, an antibiotic, and a neurohormonally active agent.

20. A system as in claim 16, wherein the acute kidney injury is caused at least in part by a kidney tumor or cancer comprising a renal cell carcinoma, and the therapeutic agent comprises a member selected from the group consisting of a vasodilator, an antioxidant, an anti inflammatory agent, a cytokine, and a neurohormonally-active agent.

21. A method for treating a patient suffering from acute kidney injury, comprising:
placing a bifurcated renal artery infusion catheter within the abdominal aorta of the patient, the bifurcated infusion catheter having a first renal delivery member with a first port and a second renal delivery member with a second port;
placing the first renal delivery member within a first renal artery of the patient, and placing the second renal delivery member within a second renal artery of the patient; and
delivering an amount of a therapeutic agent from a therapeutic agent source through the bifurcated renal artery infusion catheter and into the first and second renal arteries via the first and second ports, respectively,
wherein the acute kidney injury is characterized by at least one condition selected from the group consisting of an absolute increase in serum creatinine of more than 0.3 mg/dl within 48 hours, an increase in serum creatinine by at least 50% over baseline within 48 hours, and a decrease in urine output to 0.5 ml per kilogram of body weight or less per hour persisting for at least 6 hours.
